(19) United States
(12) Patent Application Publication
(10) Pub. No.: US 2004/0259959 A1
(43) Pub. Date: Dec. 23, 2004

(54) Method and pharmaceutical preparation for reducing the activity of cells

(76) Inventors: Yoav Sharoni, Omer (IL); Joseph Levy, Omer (IL)

Correspondence Address: BROWDY AND NEIMARK, P.L.L.C., 624 NINTH STREET, NW, SUITE 300, WASHINGTON, DC, 20001-5303, US

(21) Appl. No.: 10/495,213

(22) PCT Filed: Nov. 5, 2002
(86) PCT No.: PCT/IL02/00876

(30) Foreign Application Priority Data
Nov. 21, 2001 (IL) 146449

(51) Int. Cl. A61K 31/12; A61K 31/015

(52) U.S. Cl. 514/763; 514/690

(57) ABSTRACT

A method for reducing the activity of a cancer cell, comprising administering to a subject in need thereof a composition comprising of lycopene and an additional caroteniod.

### FIELD OF THE INVENTION

The present invention relates to a method of reducing the activity of cells, which utilizes pharmaceutical preparations comprising lycopene, beta-carotene, astaxanthin, cataxanthin, zeaxanthin, lutein, phytoene and phytofluene or mixtures thereof. The invention further relates to the inhibition of the growth of cancer cells using a composition comprising of lycopene beta-carotene, astaxanthin, cataxanthin, zeaxanthin, lutein, phytoene and phytofluene or mixtures thereof, as the anticancer active agent.

### BACKGROUND OF THE INVENTION

Lycopene, beta-carotene, astaxanthin, cataxanthin, zeaxanthin, lutein, phytoene and phytofluene are carotenoids which occur naturally in vegetables and fruit, mostly in yellow/red fruits and vegetables. The health benefits of carotenoid has long been recognized, particularly as anti-cancer and cancer preventing agents. In 1982 Mathews-Roth (Oncology, 39(1), 33-7, 1982) reported that phytoene was ineffective in treating dimethylbenz(a)anthracene(DMBA)/UV-B induced tumors in mice, but mice treated with phytoene developed fewer UV-B induced tumors than the control mice. The anti-cancer activity of phytoene has been implied in a report by Nishino (Mutat. Res., 402(1-2):159-63 1998; J. Cell Biochem. Suppl., 27, 86-91, 1997). Nishino applied new methods in biotechnology, wherein mammalian cells producing phytoene displayed resistance to H-ras-induced cell transformation, which may be interpreted as anti-cancer activity. The anti-cancerous activity of lycopene is known and has been disclosed in U.S. Pat. No. 5,827,900. It is known the anti-cancerous and cancer-preventive activity of lycopene are dependent on the concentration of lycopene in the serum, and that said in-vivo activities are limited by the maximum physiological serum levels that may be reached even by high supplementation of lycopene. Serum levels of subjects receiving high supplementation were reported to reach a plateau at serum concentrations of about 1-1.2 μM wherein in-vitro experiments have shown improved activity with the increase of concentration even up to levels of 4 μM, which cannot be achieved in-vivo.

Accordingly, there is a long felt need for a method for increasing the anti-cancer and cancer prevention in-vivo activity of lycopene.

It is therefore a purpose of the present invention to provide a method for improving the anti-cancer and cancer preventing activity of lycopene.

It is another purpose of the present invention to provide a composition comprising, inter alia, lycopene, which has improved anti-cancer and cancer preventing activity over compositions containing only lycopene as an active ingredient.

Other objects of the invention will become apparent as the description proceeds.

### SUMMARY OF THE INVENTION

It has now been most surprisingly found, and this is an object of the invention, that lycopene together with one or more carotenoids selected from among a group comprising of beta-carotene, astaxanthin, cataxanthin, zeaxanthin, lutein, phytoene and phytofluene or mixtures thereof, display synergistic activity in reducing the overall activity of cells, both in vitro and in vivo.

It has further been found, and this is another object of the invention, that mixtures of lycopene with one or more carotenoid selected from among a group comprising of beta-carotene, astaxanthin, cataxanthin, zeaxanthin, lutein, phytoene and phytofluene or mixtures thereof, can be used as a synergistic active agent for the inhibition and prevention of growth of cancer cells.

It has further been found, and this is another object of the invention, that mixtures of lycopene with one or more carotenoid selected from among a group comprising of beta-carotene, astaxanthin, cataxanthin, zeaxanthin, lutein, phytoene and phytofluene or mixtures thereof, can be used effectively to inhibit and prevent the growth of certain particularly aggressive cancer cells.

It has further been found, and this is still another object of the invention, that lycopene mixed with one or more carotenoid selected from among a group comprising of beta-carotene, astaxanthin, cataxanthin, zeaxanthin, lutein, phytoene and phytofluene or mixtures thereof, can be used effectively to reduce cancer cell count and tumors size.

It is an object of the invention to provide a method for reducing the activity of a cell, which method comprises the use of a mixture of lycopene with one or more carotenoid selected from among a group comprising of beta-carotene, astaxanthin, cataxanthin, zeaxanthin, lutein, phytoene and phytofluene or mixtures thereof, as the active ingredient

It is another object of the invention to provide cancer growth-inhibiting and cancer preventive composition, comprising lycopene and one or more carotenoid selected from among a group comprising of beta-carotene, astaxanthin, cataxanthin, zeaxanthin, lutein, phytoene and phytofluene or mixtures thereof, as active ingredients.

It is a further object of the invention to provide anti-cancer compositions based on a mixture of lycopene with one or more carotenoid selected from among a group comprising of beta-carotene, astaxanthin, cataxanthin, zeaxanthin, lutein, phytoene and phytofluene or mixtures thereof, which can be relatively well tolerated by a patient in need thereof.

It is still another object of the invention to provide a method of treatment useful for a variety of cancer patients, based on a mixture of lycopene with one or more carotenoid selected from among a group comprising of beta-carotene, astaxanthin, cataxanthin, zeaxanthin, lutein, phytoene and phytofluene or mixtures thereof, as the active material.

Other objects and advantages of the invention will become apparent as the description proceeds.

In one aspect, therefore, the invention is directed to a method of reducing the activity of a cell, comprising administering to the cell of a subject in need thereof, directly or systemically, a cell activity-reducing effective amount of a mixture comprising of lycopene and one or more carotenoid selected from among a group comprising of beta-carotene, astaxanthin, cataxanthin, zeaxanthin, lutein, phytoene and phytofluene or mixtures thereof.

According to a preferred embodiment of the invention, the cells the activity of which it is desired to inhibit are cancer cells. Thus, in another aspect, the invention is directed to a method of inhibiting the growth of cancer cells which comprises administering to a subject in need thereof a growth-inhibiting effective amount of a mixture comprising of lycopene one or more carotenoid selected from among a group comprising of beta-carotene, astaxanthin, cataxanthin, zeaxanthin, lutein, phytoene and phytofluene or mixtures thereof.

According to a preferred embodiment of the invention, there is provided a method for preventing the development of cancer by administering to a subject an effective amount of mixture comprising of lycopene and one or more carotenoid selected from among a group comprising of beta-carotene, astaxanthin, cataxanthin, zeaxanthin, lutein, phytoene and phytofluene or mixtures thereof.

It has been found that cancer cell proliferation decreased dramatically in the breast cancer cell sample treated with a complex of the tomato carotenoids phytoene, phytofluene and lycopene, all at low concentrations.

It was also found that breast cancer cells treated with lycopene alone at the same low concentration (0.3 μM) showed no decrease in proliferation. The same dramatic inhibition of cancer cells achieved by the combination of tomato phytonutrients can be obtained when using lycopene alone, but only in the higher concentrations of 2-3 μM. However, since such high lycopene levels in the blood are not obtainable in humans, high supplemental therapeutic doses of lycopene alone will not provide practical benefit to cancer patients.

### DESCRIPTION OF THE FIGURES

The word phytoene in all of the figures means a mixture of phytoene and phytofluene. “AST” denotes astaxanthin, “LYC” denotes lycopene, “Phyto” denotes phytoene, “β” denotes β-carotene.

FIG. 1. Lycopene enhances the inhibitory effects of astaxanthin on mammary cancer cell proliferation. MCF-7 mammary cancer cell were preincubated for 24 hours in 0.5% serum and then growth ([³H]thymidine incorporation) was stimulated by 3% serum. Stimulated cells were treated for 3 days with the indicated concentrations of the astaxanthin alone or in combination with 0.3 μM lycopene (FIG. 1A). The high concentrations of lycopene and astaxanthin (FIG. 1B) are indicated. Data presented are the mean±SE of 2-3 independent experiments performed in quadruplicates.

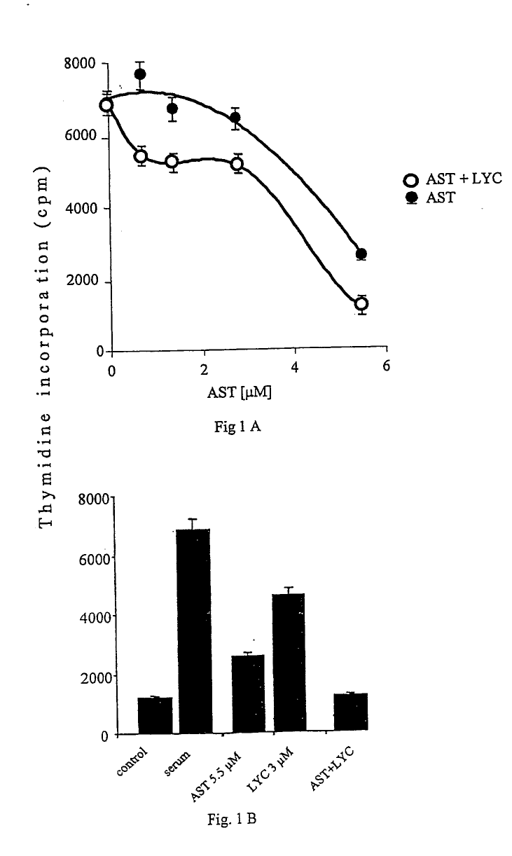

FIG. 2. Lycopene enhances the inhibitory effects of phytoene and phytofluene on DHT induced prostate cancer cell proliferation. LNCaP prostate cancer cell were preincubated for 24 hours in 1.5% serum and then growth ([³]thymidine incorporation) was stimulated by addition of 10⁻⁹ dihydrotestosterone (DHT). Stimulated cells were treated for 3 days with the indicated concentrations of the phytoene and phytofluene (phytoene) mixture alone or in combination with 0.3 μM Lycopene (plus lycopene) (FIG. 2A). The high concentrations of lycopene and phytoene plus phytofluene (FIG. 2B) are indicated. Data presented are the mean±SE of 2-3 independent experiments performed in quadruplicates.

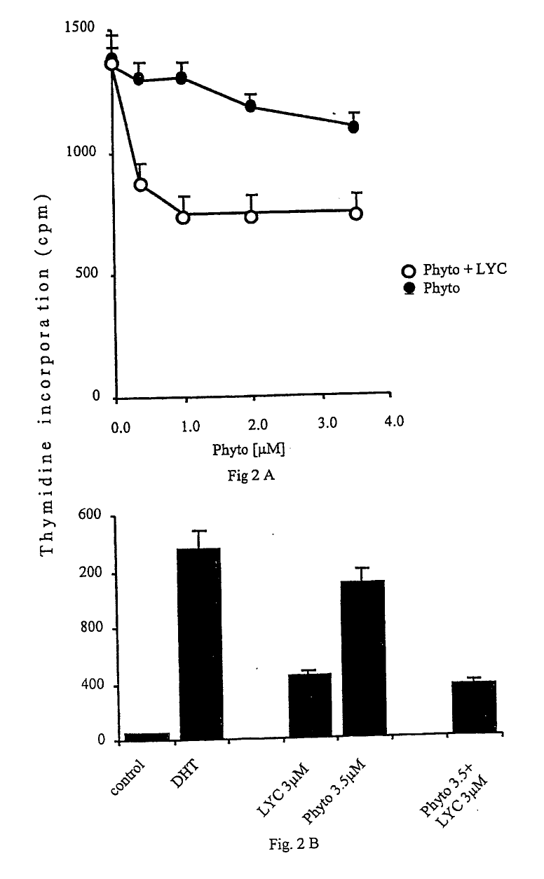

FIG. 3. Lycopene enhances the inhibitory effects of beta-carotene on DHT induced prostate cancer cell proliferation. LNCaP prostate cancer cell were preincubated for 24 hours in 1.5% serum and then growth ([³]thynidine incorporation) was stimulated by addition of 10⁻⁹ M dihydrotestosterone DHT). Stimulated cells were treated for 3 days with the indicated concentrations of the beta-carotene alone or in combination with 0.3 μM lycopene (FIG. 3A). The high concentrations of lycopene and beta-carotene (FIG. 3B) are indicated. Data presented are the mean±SE of 2-3 independent experiments performed in quadruplicates.

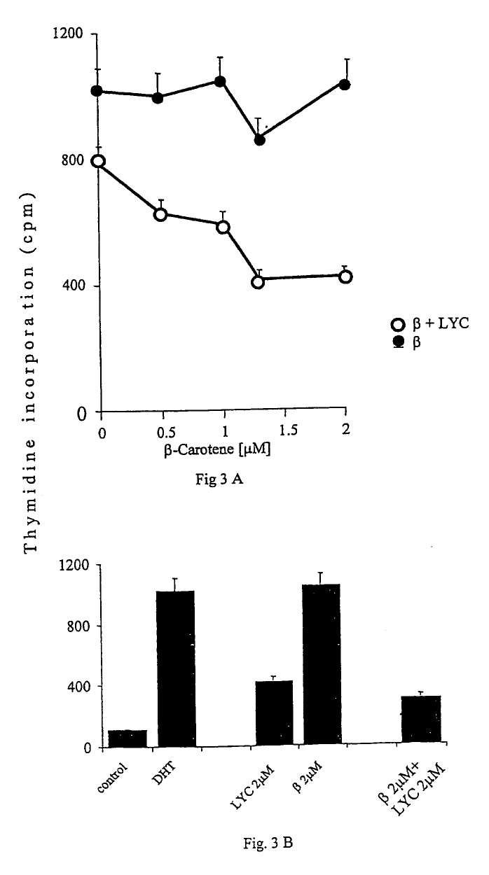

FIG. 4. Lycopene enhances the inhibitory effects of phytoene and phytofluene on mammary cancer cell proliferation. MCF-7 mammary cancer cell were preincubated for 24 hours in 0.5% serum and then growth ([³H]thymidine incorporation) was stimulated by 3% serum. Stimulated cells were treated for 3 days with the indicated concentrations of the phytoene and phytofluene (phytoene) mixture alone or in combination with 0.3 μM lycopene (FIG. 4A). The high concentrations of lycopene and phytoene plus phytofluene (FIG. 4B) are indicated. Data presented are the mean±SE of 2-3 independent experiments performed in quadruplicates.

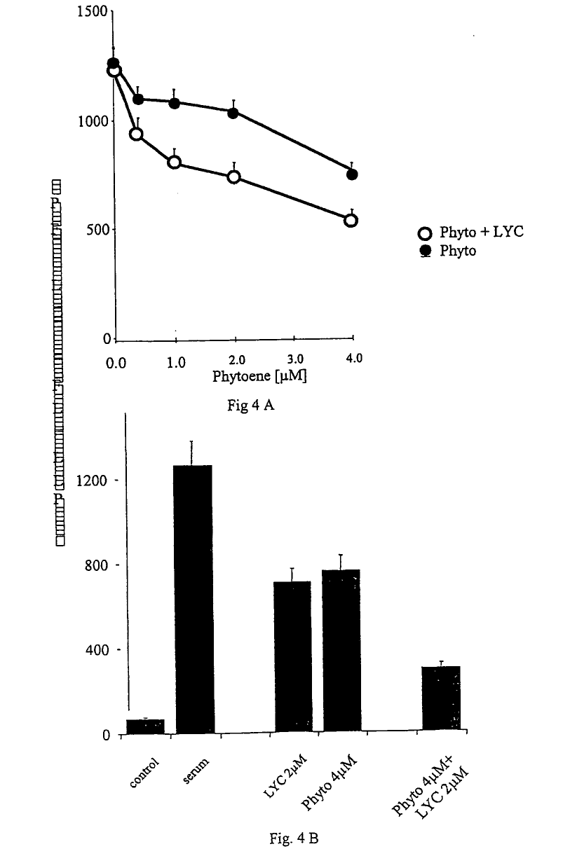

FIG. 5. Lycopene enhances the inhibitory effects of beta-carotene on mammary cancer cell proliferation. MCF-7 mammary cancer cell were preincubated for 24 hours in 0.5% serum and then growth ([³H]thymidine incorporation) was stimulated by 3% serum. Stimulated cells were treated for 3 days with the indicated concentrations Of the beta-carotene alone or in combination with 0.3 μM lycopene (FIG. 5A). The high concentrations of lycopene and beta-carotene (FIG. 5B) are indicated. Data presented are the mean±SE of 2-3 independent experiments performed in quadruplicates.

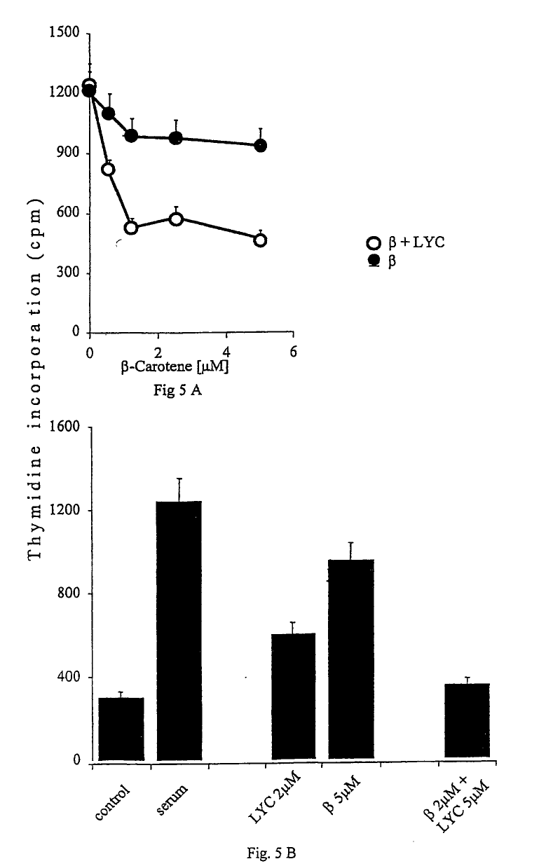

### DETAILED DESCRIPTION OF A PREFERRED EMBODIMENT OF THE INVENTION

The following description is illustrative of embodiments of the invention. The following description is not to be construed as limiting, it being understood that the skilled person may carry out many obvious variations to the invention.

Throughout the description, percentages of components are by weight, unless specifically noted differently. The term “lycopene” throughout the application refers to lycopene from natural sources such as vegetables, fruits, other plant matter and fungal sources, and synthetic lycopene. It is intended that in the present context, the cancer-growth inhibiting activity also encompasses cancer preventive activity, i.e. therapeutic and preventive activity, both of which are provided by the present invention.

According to a particular embodiment of the method of the present invention there is provided a method for inhibiting the activity of cancer cells and preventing the development of cancer, comprising administering to a subject in need thereof, an inhibitory or preventive effective amount of a composition containing lycopene and one or more carotenoid selected from among a group comprising of beta-carotene, astaxanthin, cataxanthin, zeaxanthin, lutein, phytoene and phytofluene or mixtures thereof, wherein the ratio between the lycopene and each of the other carotenoids or mixtures thereof are in the range of about 10:0.3 to 1:1, preferably 6:1. According to a preferred embodiment of the invention the composition used in the present method comprises of lycopene, phytoene and phytofluene in a ratio of about 10:0.67:0.6.

According to yet a further particular embodiment of the method of the present invention there is provided a method for inhibiting the activity of cancer cells and preventing the development of cancer, comprising administering to a subject in need thereof, an inhibitory or preventive effective amount of a composition containing 5 to 40 mg lycopene and 0.15 to 6 mg of one or more carotenoid selected from among a group comprising of beta-carotene, astaxanthin, cataxanthin, zeaxanthin, lutein, phytoene and phytofluene or mixtures thereof. Accordingly, said administration may be by daily single or multiple dosages, wherein multiple dosage is preferred.

In yet a further embodiment of the present method 80 to 670 mg of a composition containing 5% to 15% lycopene and 0.3% to 10% of one or more carotenoid selected from among a group comprising of beta-carotene, astaxanthin, cataxanthin, zeaxanthin, lutein, phytoene and phytofluene or mixtures thereof, is administered to a subject, for the inhibition of cancer cell activity or the prevention of cancer.

In yet a further embodiment of the present method 250 mg of a composition containing 5% to 15% lycopene, 0.3% to 3% phytoene and 0.3% to 4% phytofluene are administered. More preferably said composition contains 6% lycopene, 0.5% phytoene and 0.55% phytofluene is administered to a subject, for the inhibition of cancer cell activity or the prevention of cancer. Said composition may further comprise other conventional anti-cancer agents and additives.

Preferably administration is 15 mg lycopene, 1.2 mg phytoene and 1.4 mg phytofluene, twice daily.

Administration according to the method of the present invention may be according to known methods in the art, e.g. oral, topical, rectal, subcutaneous, intravenous or intramuscular injection.

According to a further embodiment of the present invention there is provided a synergistic composition containing lycopene and one or more carotenoid selected from among a group comprising of beta-carotene, astaxanthin, cataxanthin, zeaxanthin, lutein, phytoene and phytofluene or mixtures thereof, wherein the ratio between the lycopene and other carotenoids or mixtures thereof are in the range of about 10:0.3 to 1:1, preferably 6:1. More preferably the composition comprises of lycopene, phytoene and phytofluene in a ratio of about 10:0.3:0.2 to 1:1:1.

According to a further embodiment of the composition of the present invention there is provided a composition containing 3% to 15% lycopene and 0.3% to 15% of one or more carotenoid selected from among a group comprising of beta-carotene, astaxanthin, cataxanthin, zeaxanthin, lutein, phytoene and phytofluene or mixtures thereof. Preferably, 5% to 8% lycopene, 0.4% to 1% phytoene and 0.4% to 1% phytofluene. More preferably 6% lycopene, 0.5% phytoene and 0.5% phytofluene.

The compositions of the present invention which are also used in the method of the present invention may further contain a further ingredient selected from among a group comprising of conventional pharmaceutical, conventional anti-cancer agents, additives, excepients, adjuvants and carriers. The compositions according to the present invention may be formulated in various dosage forms. Illustrative, non-limiting examples of such dosage forms are soft and hard capsules, gel-cap, pellet, soft gel capsule, tablet, granules, grains, powder, liquid formulation, e.g., as suspensions. For parenteral administration other commonly employed forms, such as injections, drops, suppositories, etc. The formulations may further be slow-release form.

The composition of the present invention has been found to be surprisingly active in inhibiting the growth of a variety of cancer cells and preventing the development of cancer. The invention is not to be construed as being limited to any particular type of cancer cell. Illustrative and non-limiting examples of such cancer cells the growth of which can be inhibited according to the method of the invention are: breast cancer, endometrial cancer, prostatic cancer, ovarian cancer, lung cancers (small and non-small cell types), melanomas, bladder cancer, pancreatic cancer, gastric cancer, hepatic cancer, leukemias, glioblastoma, neuroblastoma and other brain tumors, head and neck cancers and cervical cancer.

According to yet a further aspect of the present method, administration is via the addition of a composition containing lycopene and one or more carotenoid selected from among a group comprising of beta-carotene, astaxanthin, cataxanthin, zeaxanthin, lutein, phytoene and phytofluene or mixtures thereof, to food-stuff, nutritional supplements, functional foods or beverages.

The present invention presents the following advantages:

1. The anti-cancer and cancer prevention activity of lycopene is improved by creating a synergistic effect with one or more carotenoid selected from among a group comprising of beta-carotene, astaxanthin, cataxanthin, zeaxanthin, lutein, phytoene and phytofluene or mixtures thereof.

2. The present method achieves the anti-cancer and cancer prevention effect of high lycopene concentrations which are unattainable in-vivo.

3. The present method provides a safe method with little or no side effects, which are associated with the use of conventional anti-cancer pharmaceuticals.

While embodiments of the invention have been described by way of illustration, it will be apparent that the invention may be carried out with many modifications, variations and adaptations, without departing from its spirit or exceeding the scope of the claims.

1. A method for reducing the activity of a cancer cell, comprising administering to a subject in need thereof a composition comprising a cancer cell activity-reducing amount of lycopene and an additional carotenoid.

2. A method for reducing the activity of a cancer cell, comprising administering to a subject in need thereof a composition comprising a cancer cell activity-reducing amount of lycopene and a carotenoid selected from the group consisting of beta-carotene, astaxanthin, cataxanthin, zeaxanthin, lutein, phytoene, phytofluene and mixtures thereof.

3. A method of inhibiting the growth and preventing the development of cancer cells, comprising administering to a subject in need thereof a composition comprising of a growth inhibiting effective amount of lycopene and a carotenoid selected from the group consisting of beta-carotene, astaxanthin, cataxanthin, zeaxanthin, lutein, phytoene, phytofluene and mixtures thereof, said composition containing a growth-inhibiting effective amount of lycopene.

4. A method of inhibiting the growth and preventing the development of cancer cells, wherein the cancer cells, the growth of which is to be inhibited, are selected from the group consisting of breast cancer, endometrial cancer, prostatic cancer, ovarian cancer, lung cancers (small and non-small cell types), melanomas, bladder cancer, pancreatic cancer, gastric cancer, hepatic cancer, leukemias, glioblastoma, neuroblastoma and other brain tumors, head cancer, neck cancer, and cervical cancer, comprising
administering to a subject in need thereof a composition comprising a cancer-growth inhibiting amount of lycopene and a carotenoid selected from the group consisting of beta-carotene, astaxanthin, cataxanthin, zeaxanthin, lutein, phytoene, phytofluene and mixtures thereof, said composition containing a growth-inhibiting effective amount of lycopene.

5. A method according to claim 4 wherein said composition is is in a form suitable for administration by injection or orally.

6. A method of treating a cancer patient having mammary cancer, endometrial cancer, prostatic cancer, ovarian cancer, lung cancers (small and non-small cell types), melanomas, bladder cancer, pancreatic cancer, gastric cancer, hepatic cancer, leukemias, glioblastoma, neuroblastoma and other brain tumors, and cervical cancer, comprising
administering to said patient a pharmaceutically-effective amount of a composition comprising lycopene and a carotenoid selected from the group consisting of beta-carotene, astaxanthin, cataxanthin, zeaxanthin, lutein, phytoene, phytofluene and mixtures thereof, in a cancer cell growth inhibiting effective amount as the active ingredients for inhibiting the growth of cancer cells.

7. A method for preventing the development of cancer comprising administering to a subject in need thereof a cancer-preventing effective amount of a composition containing lycopene and a carotenoid selected from the group consisting of beta-carotene, astaxanthin, cataxanthin, zeaxanthin, lutein, phytoene phytofluene and mixtures thereof.

8. A cancer-cell-activity inhibiting composition comprising a cancer-cell-activity inhibiting amount of lycopene and an additional carotenoid.

9. A cancer-cell-activity inhibiting composition according to claim 8 comprising said lycopene and wherein said additional carotenoid is selected from the group consisting of beta-carotene, astaxanthin, cataxanthin, zeaxanthin, lutein, phytoene, phytofluene and mixtures thereof.

10. A cancer-cell-activity inhibiting composition according to claim 9 wherein said additional carotenoid comprises beta-carotene.

11. A cancer-cell-activity inhibiting composition according to claim 9 wherein said additional carotenoid comprises astaxanthin.

12. A cancer-cell-activity inhibiting composition according to claim 9 wherein said additional carotenoid comprises cataxanthin.

13. A cancer-cell-activity inhibiting composition according to claim 9 wherein said additional carotenoid comprises zeaxanthin.

14. A cancer-cell-activity inhibiting composition according to claim 9 wherein said additional carotenoid comprises lutein.

15. A cancer-cell-activity inhibiting composition according to claim 9 wherein said additional carotenoid comprises phytoene.

16. A cancer-cell-activity inhibiting composition according to claim 9 wherein said additional carotenoid comprises phytofluene.

17. A composition according to claim 8, further comprising a pharmaceutical additive, excepient, adjuvant or carrier.

18. A composition effective in preventing the development of cancers comprising lycopene and a carotenoid selected from the group consisting of beta-carotene, astaxanthin, cataxanthin, zeaxanthin, lutein, phytoene, phytofluene and mixtures thereof, said composition being in a unit dosage form and containing an amount sufficient of said lycopene and carotenoid effective for inhibiting the development of at least some cancers.

19. A composition according to claim 8 in an oral, topical, rectal or liquid dosage form.

20. A method for reducing the activity of a cancer cell, comprising administering to a subject in need thereof a composition comprising a cancer cell activity reducing amount of the composition of claim 8, wherein the additional carotenoid is beta-carotene.

21. A method for reducing the activity of a cancer cell, comprising administering to a subject in need thereof a composition comprising a cancer cell activity reducing amount of the composition of claim 8, wherein the additional carotenoid is astaxanthin.

22. A method for reducing the activity of a cancer cell, comprising administering to a subject in need thereof a composition comprising a cancer cell activity reducing amount of the composition of claim 8, wherein the additional carotenoid is cataxanthin.

23. A method for reducing the activity of a cancer cell, comprising administering to a subject in need thereof a composition comprising a cancer cell activity reducing amount of the composition of claim 8, wherein the additional carotenoid is zeaxanthin.

24. A method for reducing the activity of a cancer cell, comprising administering to a subject in need thereof a composition comprising a cancer cell activity reducing amount of the composition of claim 8, wherein the additional carotenoid is lutein.

25. A method for reducing the activity of a cancer cell, comprising administering to a subject in need thereof a composition comprising a cancer cell activity reducing amount of the composition of claim 8, wherein the additional carotenoid is phytoene.

26. A method for reducing the activity of a cancer cell, comprising administering to a subject in need thereof a composition comprising a cancer cell activity reducing amount of the composition of claim 8, wherein the additional carotenoid is phytofluene.
